(19) United States
(12) Patent Application Publication
(10) Pub. No.: US 2023/0079539 A1
(43) Pub. Date: Mar. 16, 2023

(54) METHODS FOR RAPID CLONING AND EXPRESSION OF HLA CLASS I CELLS

(71) Applicant: Board of Regents, The University of Texas System, Austin, TX (US)

(72) Inventors: Hiroki TORIKAI, Houston, TX (US); Shan ZONG, Houston, TX (US)

(73) Assignee: Board of Regents, The University of Texas System, Austin, TX (US)

(21) Appl. No.: 17/929,013

(22) PCT Filed: Feb. 10, 2021
(86) PCT No.: PCT/US2021/017455

§ 371 Date: Aug. 10, 2022

Related U.S. Application Data
(60) Provisional application No. 62/972,210, filed on Feb. 10, 2020.

(51) Int. Cl. A61K 39/00; C12N 5/071; C12N 15/86; C12N 15/11; C12N 9/22

(52) U.S. Cl. CPC A61K 39/0011; C12N 5/0686; C12N 15/86; C12N 15/11; C12N 9/22; C12N 2501/24; C12N 2501/25; C12N 2501/2302; C12N 2501/2315; C12N 2502/99; C12N 2310/20; C12N 2800/80; C12N 2740/15043; A61K 2039/5158

(57) ABSTRACT

The present disclosure provides methods of generating HLA class-I null cells that can be used for expression of exogenous HLA genes and presentation of antigens, such tumor neoantigens. Method for using HLA class-I null cells from selecting, stimulating and propagating immune effector cells are also provided.

This application claims the benefit of U.S. Provisional Patent Application No. 62/972,210, filed Feb. 10, 2020, which is incorporated herein by reference in its entirety.

The sequence listing that is contained in the file named “UTFCP1486WO_ST25.txt”, which is 3 KB (as measured in Microsoft Windows) and was created on Feb. 10, 2021, is filed herewith by electronic submission and is incorporated by reference herein.

### BACKGROUND

### 1. Field

The present invention relates generally to the field of molecular biology and medicine. More particularly, it concerns methods for rapid cloning and construction of HLA class I cells with expression of an HLA class I of interest.

### 2. Description of Related Art

There are two lines of evidence that the immune system within the tumor microenvironment can correlate with clinical outcome (Sorrentino 2016; Zernecke and Preissner, 2016; and Wang et al., 2018). Firstly, infiltration of T cells, especially CD8+ T cells, is positively correlated with favorable outcome for many types of cancer (Wang et al., 2019). Secondly, therapeutic immune checkpoint blockade against CTLA-4 or PDL1/PD1 inhibit signals in tumor infiltrating lymphocytes (TIL) and results in anti-tumor effects in subsets of patients (Yu et al., 2017). TILs typically recognize neoantigens derived from tumor cell-specific mutations and expressed on tumor cells as peptides in the context of human leukocyte antigens (HLA) (Lee et al., 2019). This is supported by clinical findings demonstrating that the success of immune checkpoint blockade therapy correlates with high mutation loads in tumor cells (Wang et al., 2018; Lisabeth et al., 2013; Boyd et al., 2014; and Pasquale, 2010). A role for neoantigen-specific TIL as the mediators of immune checkpoint blockade is also supported by the observation that CD8+PD1+ T cells are enriched in the tumor microenvironment (Jing et al., 2016 and Lu et al., 2014).

These clinical observations provide a blueprint to improve immunotherapy based on the adoptive transfer of neoantigen-specific T cells with a diversity of αβ T-cell receptors (TCRαβ) (Lee et al., 2016). The ex vivo numeric expansion of TIL has shown promise for the treatment of metastatic melanoma and on occasion other solid tumors (Hsu et al., 2018; Badve and Nakshatri, 2012; Moser et al., 2018; and Arvanitis and Davy, 2018). However, an inherent limitation with TIL-based immunotherapy is that ex vivo culture and numeric expansion typically leads to clonal and/or oligoclonal expansion of terminally differentiated T cells. Together, these clinical data suggest that there may be an advantage with the administration of “young” T cells sourced from peripheral blood that are genetically modified to be neoantigen-specific. However, there is an unmet need for neoantigen-specific T cells, particularly of artificial antigen presenting cells that are HLA matched to the patient.

### SUMMARY

In a first embodiment, the present disclosure provides an HLA class-I_{neg} cell population, said cells having no expressible HLA class-I protein and having a disruption in the HLA-A, HLA-B and HLA-C genes, said cells produced by CRISPR/Cas9-mediated gene disruption targeted to a consensus sequence share between HLA-A, HLA-B, and HLA-C.

In some aspects, the cells are an immortalized cell line. In some aspects the cells are human cells. In certain aspects, the cells are immortalized kidney cells. For example, the cells are 293 cells, such as 293T cells.

In certain aspects, the consensus sequence shared between HLA-A, HLA-B, and HLA-C is GGCTACTACAACCAGAGCG (SEQ ID NO:1). In some aspects, the cells have no detectable HLA class-I expression following treatment with IFNγ and/or TNFα. In further aspects, the cells are further defined as antigen presenting cells.

In some aspects, the cells comprise at least a first expression construct for an exogenous HLA class I gene. In certain aspects, the exogenous HLA class I gene is a HLA-A, HLA-B, and/or HLA-C gene. In particular aspects, the exogenous HLA class I gene is a gene from a human cancer patient. In specific aspects, the exogenous HLA class I gene was cloned from the subject using locus-specific primer pairs. In some aspects, the subject has not been HLA typed.

In certain aspects, the cells comprise at least a first expression construct for an exogenous HLA class I gene was constructed by Gibson assembly. In some aspects, the exogenous HLA class I gene expresses an HLA polypeptide that binds to cancer antigen. For example, the exogenous HLA class I gene is comprised on a plasmid vector, an episomal vector or a viral vector. In specific aspects, the exogenous HLA class I gene is comprised on lentiviral vector.

In certain aspects, the exogenous HLA class I gene expresses an HLA polypeptide that binds to cancer antigen and wherein the cells have been loaded with the cancer cell antigen that binds to the HLA polypeptide. In some aspects, the HLA polypeptide is bound to the cancer cell antigen and present at the cell surface.

In some aspects, the cells are cultured in the presence of polybrene. In particular aspects, the cells are cultured in the presence of about 1 to 20 ug/mL, 1 to 10 ug/mL, 5 to 10 ug/mL, or about 8 ug/mL of polybrene.

In additional aspects, the cells are co-cultured with immune effector cells. In some aspects, the immune effector cells are primary cells. In certain aspects, the immune effector cells comprise T-cell, NK-cells or NK/T-cells. In some aspects, the T-cell comprise CD8⁺ T-cells or CD4⁺ T-cells. In some aspects, the cells are in co-culture in medium that comprises cytokines that stimulate activation and/or growth of immune effector cells. In particular aspects, medium comprises IL-2 or IL-15. In certain aspects, the cells are an adherent cell culture. In some aspects, the cells are immobilized. In particular aspects, the cells have been inactivated.

In some aspects, the population further comprises a co-culture of the cells with cells that express a candidate TCR or candidate CAR molecule.

A further embodiment provides a method of stimulating immune effector cells comprising culturing a population of immune effector cells with antigen presenting cells in accordance with the present embodiments (e.g., an HLA class-I_{neg} cell population, said cells having no expressible HLA class-I protein and having a disruption in the HLA-A, HLA-B and HLA-C genes, said cells produced by CRISPR/Cas9-mediated gene disruption targeted to a consensus sequence share between HLA-A, HLA-B, and HLA-C), wherein the antigen presenting cells comprise an exogenous HLA class I gene that expresses an HLA polypeptide and wherein the cells have been loaded with the cancer cell antigen that binds to the HLA polypeptide.

Another embodiment provides a method of identifying an antigen effective for stimulating immune cells comprising (a) loading a population of antigen presenting cells in accordance with the present embodiments (e.g., an HLA class-I_{neg} cell population, said cells having no expressible HLA class-I protein and having a disruption in the HLA-A, HLA-B and HLA-C genes, said cells produced by CRISPR/Cas9-mediated gene disruption targeted to a consensus sequence share between HLA-A, HLA-B, and HLA-C) with a candidate antigen, wherein the antigen presenting cells comprise an exogenous HLA class I gene that expresses an HLA polypeptide; and (b) determining whether the antigen can stimulate immune effector cells.

In yet another embodiment, there is provided a method of treating a subject comprising administering an effective amount of immune effector cells that have been stimulated by the methods of the present embodiments.

A further embodiment provides a method for the generation of HLA class I_{neg} cells comprising engineering a cell to express a CRISPR-Cas9 construct targeting the consensus sequence of shared by the HLA-A, HLA-B and HLA-C genes to produce HLA class I_{neg} cells.

In some aspects, the cells are an immortalized cell line. In certain aspects, the cells are immortalized kidney cells. For example, the cells are 293 cells, such as 293T cells. In certain aspects, the cells are defined as antigen presenting cells.

In certain aspects, the consensus sequence share between HLA-A, HLA-B, and HLA-C is GGCTACTACAACCAGAGCG (SEQ ID NO:1). In some aspects, the cells are transfected with the CRISPR-Cas9 plasmid for 10-14 days. In particular aspects, the cells have no detectable HLA class-I expression following treatment with IFNγ and/or TNFα.

In additional aspects, the method further comprises selecting a cell exhibiting no detectable HLA class-I expression and growing the selected cell to produce HLA class I_{neg} cells. In some aspects, the method further comprises selecting a cell exhibiting no detectable HLA class-I expression in the presence of IFNγ and TNFα and growing the selected cell to produce HLA class I_{neg} cells.

In some aspects, the method further comprises introducing at least a first expression construct for an exogenous HLA class I gene into the cells. In some aspects, the exogenous HLA class I gene is a HLA-A, HLA-B, and/or HLA-C gene. In certain aspects, the exogenous HLA class I gene is a gene from a human cancer patient. In some aspects, the exogenous HLA class I gene was cloned from the subject using locus-specific primer pairs. In certain aspects, the subject has not been HLA typed.

In certain aspects, the at least a first expression construct for an exogenous HLA class I gene is constructed by Gibson assembly. In some aspects, the exogenous HLA class I gene expresses an HLA polypeptide that binds to cancer antigen. In some aspects, introducing the exogenous HLA class I gene comprises transfecting the cells with a plasmid vector or an episomal vector or transducing the cells with a viral vector. In some aspects, introducing the exogenous HLA class I gene comprises transducing the cells with a lentiviral vector. In certain aspects, the exogenous HLA class I gene expresses an HLA polypeptide that binds to cancer antigen, the method further comprising loading the cells with the cancer cell antigen that binds to the HLA polypeptide. In some aspects, the cells are loaded with the cancer cell antigen polypeptide. In particular aspects, the cells are loaded with a vector encoding the cancer cell antigen.

In some aspects, the cells are cultured in the presence of polybrene. For example, the cells are cultured in the presence of about 1 to 20 ug/mL, 1 to 10 ug/mL, 5 to 10 ug/mL, or about 8 ug/mL of polybrene.

In additional aspects, the method further comprises a co-culture the cells with immune effector cells. For example, the immune effector cells are primary cells. In some aspects, the immune effector cells comprise T-cell, NK-cells or NK/T-cells. In specific aspects, the T-cell comprise CD8⁺ T-cells or CD4⁺ T-cells.

In some aspects, the cells are in co-culture in a medium that comprises cytokines that stimulate activation and/or growth of immune effector cells. In particular aspects, the medium comprises IL-2 or IL-15.

In some aspects, the cells are an adherent cell culture. In certain aspects, the cells are immobilized. In some aspects, the cells have been inactivated. In additional aspects, the method further comprised a co-culture of the cells with cells that express a candidate TCR or candidate CAR molecule. In some aspects, the method further comprised selecting a candidate TCR or CAR based on binding to the cancer cell antigen.

In another embodiment, there is provided a composition comprising a CRISPR guide RNA targeting a consensus sequence shared between HLA-A, HLA-B, and HLA-C for use in the preparation of an HLA class-I_{neg} cell population in accordance with any of claims 1-31. In some aspects, the consensus sequence shared between HLA-A, HLA-B, and HLA-C is GGCTACTACAACCAGAGCG (SEQ ID NO:1).

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this DETAILED DESCRIPTION.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1E: HLA cloning and expression in HLA class I disrupted HEK293T cells. (FIG. 1A) Target for guide RNA to disrupt HLA-A, B, C expression from HEK293T cells. Guide RNA was designed to bind consensus sequences of HLA class I expressed in HEK293T cells. Target sequence (GGCTACTACAACCAGAGCG; SEQ ID NO:1) and PAM sequence (AGG) for SpCas9 are shown. (CCTGCGCGGCTACTACAACCAGAGCGAGGCC=SEQ ID NO: 9; CCTGCGCGGCTACTACAACCAGAGCGAGGAC=SEQ ID NO: 10) (FIG. 1B) Elimination of HLA class I expression from HEK293T cells. HEK293T cells were transfected with single DNA plasmid encoding guide RNA and Cas9. Three days after transfection, HEK293 T cells were stained with anti-HLA-A2 monoclonal antibody and anti-HLA-A, B, C monoclonal antibody. Numbers shown are percentage of HLA class I negative HEK293T cells. IFNγ and TNFα: Cells were cultured with 600 IU/mL of interferon-γ and 10 ng/mL of tissue-necrosis factor α for two days. (FIG. 1C) Representative agarose gel picture of HLA class I cloning from cDNA generated from PBMC. (FIG. 1D) Transfection of HLA encoding lentivirus plasmid along with helper plasmids to HEK293T cells and subsequent culture to generate HEK293T cells expressing HLA of interest. (FIG. 1E) Representative expression of cloned HLA class I on HEK293T cells. Each number represents the expression of introduced HLA on HLA class I negative HEK293T cells.

### DESCRIPTION OF ILLUSTRATIVE EMBODIMENTS

The recent success of cancer immunotherapy provides the foundation for future immunotherapy based on T cell receptors (TCRs) that recognize tumor-associated antigens, especially neoantigens derived from tumor cell specific mutation. However, a major barrier to identify and validate those neoantigen specific T cells is the lack of a high-throughput system to isolate and evaluate candidate neoantigens and corresponding TCRs that recognize them. To evaluate antigen specificity and function of TCRs in vitro, stimulator cells are needed which express patient specific HLA molecules. Due to the highly polymorphic nature of HLA, cloning and expressing an HLA of interest is a time-consuming procedure. Also, native HLAs expressed on stimulator cell lines cause promiscuous TCR mediated activation. Thus, there is an unmet need for preparing stimulator cells which express a patient's specific HLA is an important step to identify and evaluate potential tumor specific antigens, including neoantigens, for T cell receptor therapy. Accordingly, in certain embodiments, the present disclosure provides methods and compositions for the HLA class I negative cells, such as HEK293T cells. Further provided herein are methods of using the HLA class I negative cells as master stimulator cells which have minimum cross reactivity.

Currently, HLA cloning is done with allele specific primers after typing HLAs. For HLA negative stimulator cells, non-adherent cells are available (e.g., K562, 721.221, etc). However, those cells cannot be used to produce lentivirus vector. Simultaneous production of HLA-I negative HEK293T cells expressing patient derived HLA and lentivirus vector would facilitate the process of identifying tumor associated antigens and their corresponding TCRs.

Thus, additional embodiments of the present disclosure provide methods for cloning patient-specific HLA alleles, including HLA-A, HLA-B, and HLA-C alleles, such as through the use of locus specific primer pairs with patient peripheral blood mononuclear cells (PBMCs). The present primer sets can be directly used for HLA cloning without prior knowledge of HLA alleles by HLA typing. Finally, further embodiments of the present provide methods for the production of patient-specific stimulator cells by expressing the patient-specific alleles in the HLA class I negative cells produced by the present methods. The production of the patient-specific stimulator cells may comprise lentivirus vectors encoding the patient HLA alleles, such as by Gibson assembly.

Therefore, the present methods provide streamlined HLA class I cloning, stable expression in HLA-I negative HEK293T cells, and the production of lentivirus particle for identifying and validating tumor specific antigens for T cells. The HLA-I negative HEK293T cells can be used as master cells for the stimulation of TCR expressing cells.

### I. DEFINITIONS

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The term “about” means, in general, the stated value plus or minus 5%.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

“Prophylactically treating” includes: (1) reducing or mitigating the risk of developing the disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

The term “effective,” as that term is used in the specification and/or claims, means adequate to accomplish a desired, expected, or intended result. “Effective amount,” “therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Non-limiting examples of such salts include acid addition salts formed with inorganic acids such as hydrochloric acid, hydrobromic acid, sulfuric acid, nitric acid, and phosphoric acid; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, and trimethylacetic acid. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Non-limiting examples of acceptable organic bases include ethanolamine, diethanolamine, triethanolamine, tromethamine, and N-methylglucamine. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

An “allele” refers to one of two or more forms of a gene. Diploid organisms such as humans contain two copies of each chromosome, and thus carry one allele on each.

The term “homozygous” is defined as containing two of the same alleles at a particular locus. The term “heterozygous” refers to as containing two different alleles at a particular locus. A “haplotype” refers to a combination of alleles at multiple loci along a single chromosome. A haplotype can be based upon a set of single-nucleotide polymorphisms (SNPs) on a single chromosome and/or the alleles in the major histocompatibility complex.

As used herein, a “disruption” of a gene refers to the elimination or reduction of expression of one or more gene products encoded by the subject gene in a cell, compared to the level of expression of the gene product in the absence of the disruption. Exemplary gene products include mRNA and protein products encoded by the gene. Disruption in some cases is transient or reversible and in other cases is permanent. Disruption in some cases is of a functional or full-length protein or mRNA, despite the fact that a truncated or non-functional product may be produced. In some embodiments herein, gene activity or function, as opposed to expression, is disrupted. Gene disruption is generally induced by artificial methods, i.e., by addition or introduction of a compound, molecule, complex, or composition, and/or by disruption of nucleic acid of or associated with the gene, such as at the DNA level. Exemplary methods for gene disruption include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques which result in targeted gene inactivation or disruption, e.g., by induction of breaks and/or homologous recombination. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

### II. GENERATION OF PATIENT SPECIFIC HLA CLASS I ARTIFICIAL PRESENTING CELLS

Certain embodiments of the present disclosure concern the generation of HLA Class I negative cells. The methods may comprise disruption of the HLA Class I allele by using a CRISPR-Cas9 plasmid encoding a guide RNA targeting the consensus sequence in HLA Class I (SEQ ID NO:2; GGCTACTACAACCAGAGCGAGGCC).

1. CRISPR-Cas9

In some embodiments, the disruption of HLA Class I is achieved using a DNA-targeting molecule, such as a DNA-binding protein or DNA-binding nucleic acid, or complex, compound, or composition, containing the same, which specifically binds to or hybridizes to the gene. In some aspects, a ribonucleotide (RNP) comprising purified Cas9 protein in complex with a targeting gRNA may be delivered directly to the cell for rapid and highly efficient genome editing. The RNP delivery method not only has high specificity but also does not have a risk of insertional mutagenesis. RNP delivery limits exposure to genome editing reagents, reduces off-target events, drives high rates of homology-dependent repair, and can be applied to embryos to rapidly generate animal models. The RNP, when paired with a DNA donor, comprises a package that does not require the cellular environment to synthesize Cas9 and sgRNA, and ensures temporal coordination of the editing reagents. In some aspects, the Cas 9 may be Streptococcus pyogenes Cas9 expressed in E. coli from a bacterial expression vector. The sgRNA may be made by in vitro transcription (IVT) in two steps: template synthesis by assembly PCR followed by vitro transcription and purification of the sgRNA. The RNP complex may be produced by mixing Cas9 and one or more sgRNAs in an appropriate buffer. For example, a final 1.2- to 1.5-fold molar excess of sgRNA may be used by adding Cas9 to the sgRNA slowly with manual stirring. The RNP complex may be delivered by electroporation (such as a Lonza 4D Nucleofector), which generates pores in the cell membrane, allowing entry of the RNP into the cytoplasm. (DeWitt et al., Methods, 15:121-122, 2017)

In some embodiments, the disruption is carried out using one or more DNA-binding nucleic acids, such as disruption via an RNA-guided endonuclease (RGEN). For example, the disruption can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5- transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 2011/0059502, incorporated herein by reference.

2. Delivery of Nucleic Acids

In some aspects, a nucleic acid encoding the DNA-targeting molecule, complex, or combination, is administered or introduced to the cell. The nucleic acid typically is administered in the form of an expression vector, such as a viral expression vector. In some aspects, the expression vector is a retroviral expression vector, an adenoviral expression vector, a DNA plasmid expression vector, or an AAV expression vector. In some aspects, one or more polynucleotides encoding the disruption molecule or complex, such as the DNA-targeting molecule, is delivered to the cell. In some aspects, the delivery is by delivery of one or more vectors, one or more transcripts thereof, and/or one or more proteins transcribed therefrom, is delivered to the cell.

In some embodiments, the polypeptides are synthesized in situ in the cell as a result of the introduction of polynucleotides encoding the polypeptides into the cell. In some aspects, the polypeptides could be produced outside the cell and then introduced thereto. Methods for introducing a polynucleotide construct into animal cells are known and include, as non-limiting examples stable transformation methods wherein the polynucleotide construct is integrated into the genome of the cell, transient transformation methods wherein the polynucleotide construct is not integrated into the genome of the cell, and virus mediated methods. In some embodiments, the polynucleotides may be introduced into the cell by for example, recombinant viral vectors (e.g. retroviruses, adenoviruses), liposome and the like. For example, in some aspects, transient transformation methods include microinjection, electroporation, or particle bombardment. In some embodiments, the polynucleotides may be included in vectors, more particularly plasmids or virus, in view of being expressed in the cells.

In some embodiments, viral and non-viral based gene transfer methods can be used to introduce nucleic acids in mammalian cells or target tissues. Such methods can be used to administer nucleic acids encoding components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, 1992; Nabel & Feigner, 1993; Mitani & Caskey, 1993; Dillon, 1993; Miller, 1992; Van Brunt, 1988; Vigne, 1995; Kremer & Perricaudet, 1995; Haddada et al., 1995; and Yu et al., 1994.

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in (e.g., U.S. Pat. Nos. 5,049,386, 4,946,787; and 4,897,355) and lipofection reagents are sold commercially (e.g., Transfectam™ and Lipofectin™) Cationic and neutral lipids that are suitable for efficient receptor-recognition lipofection of polynucleotides include those of Feigner, WO 91117424; WO 91116024. Delivery can be to cells (e.g. in vitro or ex vivo administration) or target tissues (e.g. in vivo administration).

In some embodiments, delivery is via the use of RNA or DNA viral based systems for the delivery of nucleic acids. Viral-based systems in some embodiments include retroviral, lentivirus, adenoviral, adeno-associated and herpes simplex virus vectors for gene transfer.

In some aspects, a reporter gene which includes but is not limited to glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into the cell to encode a gene product which serves as a marker by which to measure the alteration or modification of expression of the gene product. In a further embodiment, the DNA molecule encoding the gene product may be introduced into the cell via a vector. In some embodiments, the gene product is luciferase.

3. Antigen Presenting Cells (APCs)

Antigen-presenting cells, which include macrophages, B lymphocytes, and dendritic cells, are distinguished by their expression of a particular MHC molecule. APCs of the embodiments may have no expressible endogenous HLA class-I protein and can be engineered to express HLA protein of interest. APCs internalize antigen and re-express a part of that antigen, together with the MHC molecule on their outer cell membrane. The major histocompatibility complex (MHC) is a large genetic complex with multiple loci. The MHC loci encode two major classes of MHC membrane molecules, referred to as class I and class II MHCs. T helper lymphocytes generally recognize antigen associated with MHC class II molecules, and T cytotoxic lymphocytes recognize antigen associated with MHC class I molecules. In humans the MHC is referred to as the HLA complex and in mice the H-2 complex. As used herein, the terms HLA and MHC are used interchangeably.

In some cases, APCs are useful in preparing therapeutic compositions and cell therapy products of the embodiments. For general guidance regarding the preparation and use of antigen-presenting systems, see, e.g., U.S. Pat. Nos. 6,225,042, 6,355,479, 6,362,001 and 6,790,662; U.S. Patent Application Publication Nos. 2009/0017000 and 2009/0004142; and International Publication No. WO2007/103009. The APC can be derived from any antigen presenting cell including a cell line such as, for example K562, NIH/3T3, Chinese hamster ovary (CHO), or Human Embryonic Kidney (HEK) cell line.

APC systems may comprise at least one exogenous assisting molecule. Any suitable number and combination of assisting molecules may be employed. The assisting molecule may be selected from assisting molecules such as co-stimulatory molecules and adhesion molecules. Exemplary co-stimulatory molecules include CD86, CD64 (FcγRI), 41BB ligand, and IL-21. Adhesion molecules may include carbohydrate-binding glycoproteins such as selectins, transmembrane binding glycoproteins such as integrins, calcium-dependent proteins such as cadherins, and single-pass transmembrane immunoglobulin (Ig) superfamily proteins, such as intercellular adhesion molecules (ICAMs), which promote, for example, cell-to-cell or cell-to-matrix contact. Exemplary adhesion molecules include LFA-3 and ICAMs, such as ICAM-1. Techniques, methods, and reagents useful for selection, cloning, preparation, and expression of exemplary assisting molecules, including co-stimulatory molecules and adhesion molecules, are exemplified in, e.g., U.S. Pat. Nos. 6,225,042, 6,355,479, and 6,362,001. In certain aspects, APC can express IL-15, such as membrane-bound IL-15.

In some embodiments, the presently disclosed process can be used to expand immune cells, such as T-cells, in vitro using the present APCs. The process can be readily transferred to facilities operating in compliance with current good manufacturing practice (cGMP) for clinical trials. It is understood and herein contemplated that the expansion of immune cells (such as, for example T cells, NK cells, or B cells) can occur ex vivo, in vitro, or in situ with the expansion occurring outside the subject and administration occurring after expansion.

4. T-cells

In some embodiments, the T cells are derived from the blood, bone marrow, lymph, or lymphoid organs. In some aspects, the cells are human cells. The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells (e.g., CD4⁺ and/or CD8⁺ T cells) are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, one or more of the T cell populations is enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells).

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ T cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al., 2012; Wang et al., 2012.

In some embodiments, the T cells are autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2). The cells are cultured until confluence (e.g., about 2×10⁶ lymphocytes), e.g., from about 5 to about 21 days, preferably from about 10 to about 14 days. For example, the cells may be cultured from 5 days, 5.5 days, or 5.8 days to 21 days, 21.5 days, or 21.8 days, such as from 10 days, 10.5 days, or 10.8 days to 14 days, 14.5 days, or 14.8 days.

The cultured T cells can be pooled and rapidly expanded. Rapid expansion provides an increase in the number of antigen-specific T cells of at least about 50-fold (e.g., 50-, 60-, 70-, 80-, 90-, or 100-fold, or greater) over a period of about 10 to about 14 days. More preferably, rapid expansion provides an increase of at least about 200-fold (e.g., 200-, 300-, 400-, 500-, 600-, 700-, 800-, 900-, or greater) over a period of about 10 to about 14 days.

Expansion can be accomplished by any of a number of methods as are known in the art. For example, T cells can be rapidly expanded using non-specific T cell receptor stimulation in the presence of feeder lymphocytes and either interleukin-2 (IL-2) or interleukin-15 (IL-15), with IL-2 being preferred. The non-specific T-cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as an human leukocyte antigen A1 (HLA-A1) binding peptide, in the presence of a T-cell growth factor, such as IL-2. The in vitro-induced T-cells are rapidly expanded by re-stimulation with the same antigen(s) of the cancer pulsed onto HLA-A1-expressing antigen-presenting cells. Alternatively, the T-cells can be re-stimulated with irradiated, autologous lymphocytes or with irradiated HLA-A1+ allogeneic lymphocytes and IL-2, for example.

The autologous T-cells can be modified to express a T-cell growth factor that promotes the growth and activation of the autologous T-cells. Suitable T-cell growth factors include, for example, interleukin (IL)-2, IL-7, IL-15, and IL-12. Suitable methods of modification are known in the art. See, for instance, Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, N Y, 1994. In particular aspects, modified autologous T-cells express the T-cell growth factor at high levels. T-cell growth factor coding sequences, such as that of IL-12, are readily available in the art, as are promoters, the operable linkage of which to a T-cell growth factor coding sequence promote high-level expression.

5. HLA Alleles

HLA alleles for expression in HLA class-I null xcells of the embodiments include, but are not limited to alleles for HLA-A, HLA-B, HLA-C, HLA-DR, HLA-DP or HLA-DQ. Preferable, the HLA allele is a HLSA class-I allele, such as HLA-A, HLA-B and/or HLA-C.

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Production of HLA Class I Negative Cells Expressing Patient-Specific HLA Alleles

The present studies were performed for the generation of HLA class I negative cells and the simultaneous production of lentivirus particle. For the generation of HLA class-I negative cells, HEK293T cells (human kidney cell line 293 with large T) were used. HLA class I negative HEK293T cell were generated by disrupting HLA class I expression from HEK293T cells. This was done by transient expression of CRISPR-Cas9 targeting consensus sequence of HLA class-I (HLA-A, B, and -C) expressed in HEK293T cells. Further studies validated that HLA-I neg HEK293T cells did not express HLA-A, B, C molecules on the surface (FIG. 2B).

Next, locus-specific primers were used for the rapid cloning and construction of HLA class I of interest from peripheral blood monocyte (PBMC) or tumor infiltrating lymphocytes obtained from a subject. Oligo DNA primers were designed to amplify an HLA class I coding sequence from patient's PBMC. To simplify the process, each locus specific primer pair was designed to amplify any alleles in the corresponding locus. Amplified PCR products were cloned directly cloning into the lentivirus vector plasmid by Gibson assembly and patient specific HLA constructs were generated within 3 days.

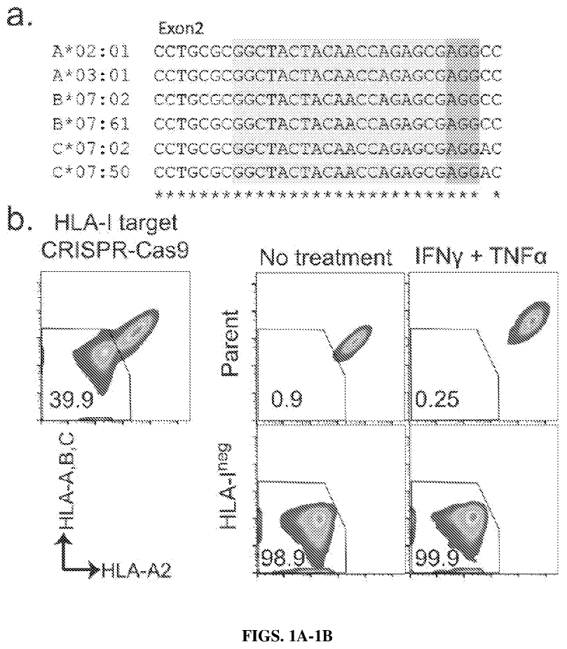

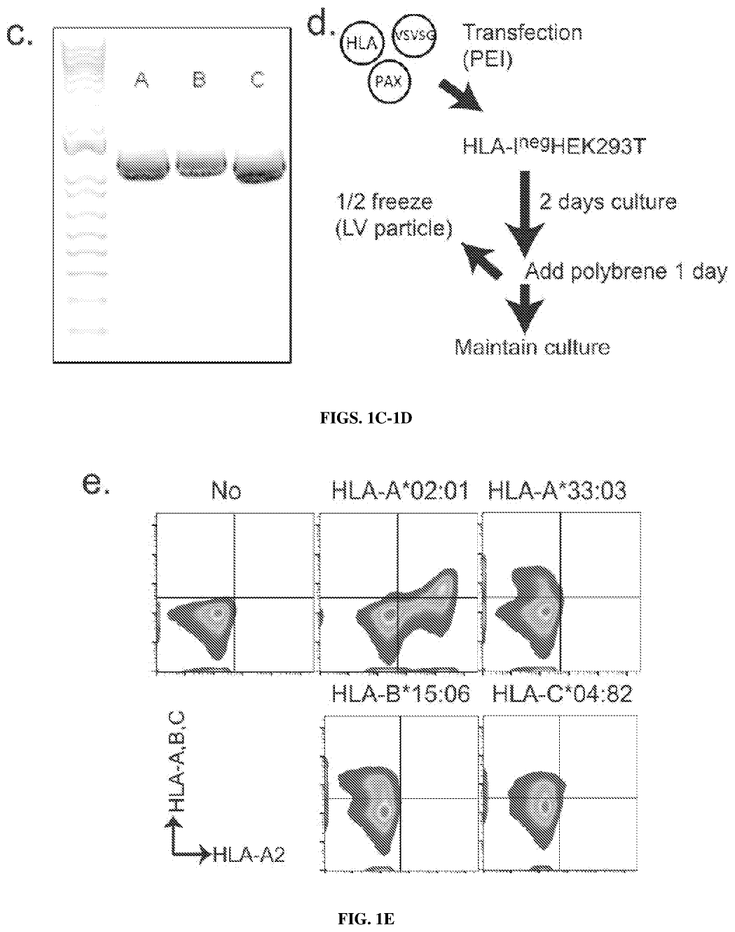

The HLA-encoding lentivirus vector plasmid was transfected into HLA-negative HEK293T cells along with lentivirus packaging plasmids by the polyethylenimine (PE1) method. Two days after transfection a half of culture medium containing lentivirus particles was collected and stored at −80° C. for future use. The remaining HLA-negative HEK293T cells in culture were added with polybrene for further culture. This addition of polybrene enabled the stable expression of the HLA of interest in HLA Class I negative HEK293T cells (FIG. 1) and those cells were further enriched with puromycin selection. These patient-derived HLA expressing HEK293T cells can be used as a stimulator cells to identify specificities of TCRs isolated from tumor tissues or PBMCs from the same patient.

### Example 2—Materials and Methods

HLA class I disruption from HEK293T cells, HLA class I cloning and expression: CRISPR: Cas9 targeting consensus sequences specific for classical HLA class I genes were generated in a DNA plasmid (px330; Addgene, Watertown, Mass., USA). DNA plasmids were transfected into HEK293T cells by polyethylenimine (PEI) method. HLA class I_{neg} HEK293T cells were isolated by negative selection using LD column (Miltenyi Biotec) with APC-conjugated mAb specific for HLA A/B/C (BD biosciences, clone G46- 2.6) and APC microbeads (Miltenyi Biotec). The sequences of HLA cloning primers were as follows; HLA-A forward: cgcagtcagtgctctagagctagcg GATTCTCCCCAGACSCCGAGG (SEQ ID NO: 3); HLA-A reverse: gtaatccagaggttgattgtcgacgc ACAAGGCAGCTGTCTCACA (SEQ ID NO: 4); HLA-B forward: cgcagtcagtgctctagagctagcg CACCCGGACTCARARTCTCCT (SEQ ID NO: 5); HLA-B reverse: gtaatccagaggttgattgtcgacgc CCTTTTCAAGCTGTGAGAG (SEQ ID NO: 6); HLA-C forward: cgcagtcagtgctctagagctagcg TTCTCCCCAGASGCCGAGATG (SEQ ID NO: 7); HLA-C reverse: gtaatccagaggttgattgtcgacgc GTCTCAGGCTTTACAAGYGA (SEQ ID NO: 8). 50ng cDNA was amplified by PCR using KOD X-tream enzyme (Millipore EMD) according to the manufacturer's instruction. PCR product was used for Gibson assembly with pCDH (EF1) MCS (PGK) Puro lentivirus plasmid (SBI SystemBiosciences, Palo Alto, Calif., USA) linearized by restriction enzyme (EcoRI and NotI) and subsequent Mung Bean Nuclease (New England Biolabs) treatment. pCDH plasmid encoding HLA was transfected into HEK293T cells along with pMD2.VSVG and psPAX2 (both purchased from Addgene) by PEI. Two days after transfection half of the culture medium was removed and stored at −80 C as lentivirus particles. Remaining culture was added with 8 ug/mL of polybrene and culture overnight. Next morning, medium was replaced with 10% FBS IMDM and further culture at 370C, 5% CO₂ incubator.

HLAneg-HEK293T cells and rapid cloning of HLA class I and class II with locus specific primer pairs: JRFTCR can be used as an antigen-discovery platform by stimulating them with antigen presenting cells. To establish stimulator cells for this assay, a system was generated to rapidly express patient's HLA molecules on HEK293T cells disrupted for endogenous HLA class I expression by CRISPR-Cas9. Other investigators have targeted β 2M by CRISPR-Cas9 to globally disrupt HLA class I expression. However, disruption of this locus will adversely affect introduced HLA class I expression unless those HLA class I construct is made as a single-chain HLA molecule that contains β₂M and antigen derived peptide (26). To avoid this complexity, a sequence was targeted which is common to all HLA class I genes expressed on HEK293T cells (HLA-A*02:01, A*03:01, HLA-B*07:02, B*07:61, HLA-C*07:02, C*07:50, FIG. 1A). Single transduction of DNA plasmid encoding the guide RNA and wild-type SpCas9 eliminated HLA class I expression from HEK293T cells up to 40%. By negative selection and single-cell cloning of the HLA_{neg} population, HLA class I_{neg} HEK293T cells were established, which does not express HLA class I even after culturing with interferon-γ and tissue-necrosis factor-α for 2 days (FIG. 1).

Next, locus-specific universal PCR-based cloning and subsequent Gibson assembly were used to generate lentivirus vector plasmids for expressing HLA molecules. Primers were designed based on a previous report (27) with some modifications to amplify coding sequences or coding sequence plus short 5′UTR sequence depending on 1 product for Gibson assembly and subsequent Sanger Sequencing authenticated the HLA-A, B, C alleles. Transfection with HLA-coding lentivirus plasmid with helper plasmids into HLA class-Ines HEK293T cells produces lentivirus particle in the culture media. Subsequent brief culture with polybrene generated HEK293T cells with sustained expression of a single HLA class I molecule on their surface (FIG. 1E).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Patent App. Pub. No. 2009/0017000
U.S. Patent App. Pub. No. 2009/0004142
U.S. Patent App. Pub. No. 2011/0059502
U.S. Pat. No. 6,225,042
U.S. Pat. No. 6,355,479
U.S. Pat. No. 6,362,001
U.S. Pat. No. 6,790,662
U.S. Pat. No. 5,049,386
U.S. Pat. No. 4,946,787
U.S. Pat. No. 4,897,355
International Publication No. WO2007/103009
International Publication No. WO2014/186469
International Publication No. WO 91117424
International Publication No. WO 91116024
International Publication No. WO2007/103009
Anderson, 1992.
Arvanitis and Davy, Genes Dev 22, 416-429, 2008.
Ausubel et al., Current Protocols in Molecular Biology, Greene Publishing Associates and John Wiley & Sons, N Y, 1994.
Badve and Nakshatri, Lancet Oncol 13, e43-48, 2012.
Boyd et al., Nat Rev Drug Discov 13, 39-62, 2014.
DeWitt et al., Methods, 15:121-122, 2017.
Dillon, 1993.
Haddada et al., 1995.
Handbook of Pharmaceutical Salts: Properties, and Use (P. H. Stahl & C. G. Wermuth eds., Verlag Helvetica Chimica Acta, 2002).
Hsu et al., Nat Commun 9, 1908, 2018.
Jing et al., Cancer Med 5, 1214-1227, 2016.
Kremer & Perricaudet, 1995.
Lee et al., Clin Cancer Res 22, 3571-3581, 2016.
Lee et al., Mol Aspects Med, 2019.
Lisabeth et al., Cold Spring Harb Perspect Biol 5, 2013.
Lu et al., Nature cell biology 16, 1105-1117, 2014.
Miller, 1992.
Mitani & Caskey, 1993
Moser et al., Int J Mol Sci 19, 2018.
Nabel & Feigner, 1993.
Pasquale, Nat Rev Cancer 10, 165-180, 2010.
Sambrook et al., Molecular Cloning: A Laboratory Manual, 3^{rd} ed., Cold Spring Harbor Press, Cold Spring Harbor, N.Y. 2001.
Sorrentino, FEBS letters 584, 2194-2200, 2010.
Terakura et al. (2012) Blood. 1:72-82, 2012.
Van Brunt, 1988.
Vigne, 1995.
Wang et al., J Immunother 35(9):689-701, 2012.
Wang et al., American journal of cancer research 9, 180-185. 2019.
Wang et al., Cancer cell 33, 752-769 e758, 2018.
Wang et al., J Biomed Sci 25, 83, 2018.
Yu et al., 1994.
Yu et al., Cell 171, 849-864 e825, 2017.
Zernecke and Preissner, Circ Res 118, 469-479, 2016.

What is claimed is:

1. An HLA class-I_{neg} cell population, said cells having no expressible HLA class-I protein and having a disruption in the HLA-A, HLA-B and HLA-C genes, said cells produced by CRISPR/Cas9-mediated gene disruption targeted to a consensus sequence shared between HLA-A, HLA-B, and HLA-C.

2. The cell population of claim 1, wherein the cells are an immortalized cell line.

3. The cell population of claim 2, wherein the cells are immortalized kidney cells.

4. The cell population of claim 3, wherein the cells are 293 cells.

5. The cell population of claim 4, wherein the cells are 293T cells.

6. The cell population of any of claims 1-5, wherein the consensus sequence shared between HLA-A, HLA-B, and HLA-C is GGCTACTACAACCAGAGCG (SEQ ID NO:1).

7. The cell population of any of claims 1-6, wherein the cells have no detectable HLA class-I expression following treatment with IFNγ and/or TNFα

8. The cell population of any of claims 1-7, further defined as antigen presenting cells.

9. The cell population of any of claims 1-8, comprising at least a first expression construct for an exogenous HLA class I gene.

10. The cell population of claim 9, wherein the exogenous HLA class I gene is an HLA-A, HLA-B, and/or HLA-C gene.

11. The cell population of claim 9, wherein the exogenous HLA class I gene is a gene from a human cancer patient.

12. The cell population of claim 11, wherein the exogenous HLA class I gene was cloned from the subject using locus-specific primer pairs.

13. The cell population of claim 11, wherein the subject has not been HLA typed.

14. The cell population of claim 9, comprising at least a first expression construct for an exogenous HLA class I gene was constructed by Gibson assembly.

15. The cell population of claim 9, wherein the exogenous HLA class I gene expresses an HLA polypeptide that binds to cancer antigen.

16. The cell population of claim 9, wherein the exogenous HLA class I gene is comprised on a plasmid vector, an episomal vector or a viral vector.

17. The cell population of claim 16, wherein the exogenous HLA class I gene is comprised on lentiviral vector.

18. The cell population of claim 9, wherein the exogenous HLA class I gene expresses an HLA polypeptide that binds to cancer antigen and wherein the cells have been loaded with the cancer cell antigen that binds to the HLA polypeptide.

19. The cell population of claim 18, wherein the HLA polypeptide is bound to the cancer cell antigen and present at the cell surface.

20. The cell population of claim 9, wherein the cells are cultured in the presence of polybrene.

21. The cell population of claim 20, wherein the cells are cultured in the presence of about 1 to 20 ug/mL, 1 to 10 ug/mL, 5 to 10 ug/mL, or about 8 ug/mL of polybrene.

22. The cell population of claim 18, further comprising a co-culture with immune effector cells.

23. The cell population of claim 22, wherein the immune effector cells are primary cells.

24. The cell population of claim 23, wherein the immune effector cells comprise T-cell, NK-cells or NK/T-cells.

25. The cell population of claim 24, wherein the T-cell comprise CD8⁺ T-cells or CD4⁺ T-cells.

26. The cell population of claim 18, wherein the cells are in co-culture in medium that comprises cytokines that stimulate activation and/or growth of immune effector cells.

27. The cell population of claim 26, wherein medium comprises IL-2 or IL-15.

28. The cell population of any of claims 1-27, wherein the cells are an adherent cell culture.

29. The cell population of any of claims 1-28, wherein the cells are immobilized.

30. The cell population of any of claims 1-29, wherein the cells have been inactivated.

31. The cell population of claim 18, further comprising a co-culture of the cells with cells that express a candidate TCR or candidate CAR molecule.

32. A method of stimulating immune effector cells comprising culturing a population of immune effector cells with antigen presenting cells in accordance with any one of claims 1-33, wherein the antigen presenting cells comprise an exogenous HLA class I gene that expresses an HLA polypeptide and wherein the cells have been loaded with the cancer cell antigen that binds to the HLA polypeptide.

33. A method of identifying an antigen effective for stimulating immune cells comprising:
(a) loading a population of antigen presenting cells in accordance with any one of claims 1-33 with a candidate antigen, wherein the antigen presenting cells comprise an exogenous HLA class I gene that expresses an HLA polypeptide; and
(b) determining whether the antigen can stimulate immune effector cells.

34. A method of treating a subject comprising administering an effective amount of immune effector cells that have been stimulated in accordance with claim 32.

35. A method for the generation of HLA class I_{neg} cells comprising engineering a cell to express a CRISPR-Cas9 construct targeting a consensus sequence shared by the HLA-A, HLA-B and HLA-C genes to produce HLA class I_{neg} cells.

36. The method of claim 35, wherein the cells are an immortalized cell line.

37. The method of claim 36, wherein the cells are immortalized kidney cells.

38. The method of claim 37, wherein the cells are 293 cells.

39. The method of claim 38, wherein the cells are 293T cells.

40. The method of any of claims 35-39, wherein the consensus sequence shared between HLA-A, HLA-B, and HLA-C is GGCTACTACAACCAGAGCG (SEQ ID NO:1).

41. The method of any of claims 35-40, wherein the cells are transfected with the CRISPR-Cas9 plasmid for 10-14 days.

42. The method of any of claims 35-41, wherein the cells have no detectable HLA class-I expression following treatment with IFNγ and/or TNFα.

43. The method of any of claims 35-42, further comprising selecting a cell exhibiting no detectable HLA class-I expression and growing the selected cell to produce HLA class I_{neg} cells.

44. The method of any of claims 35-43, further comprising selecting a cell exhibiting no detectable HLA class-I expression in the presence of IFNγ and TNFα and growing the selected cell to produce HLA class I_{neg} cells.

45. The method of any of claims 35-44, wherein the cells are defined as antigen presenting cells.

46. The method of any of claims 35-45, further comprising introducing at least a first expression construct for an exogenous HLA class I gene into the cells.

47. The method of claim 46, wherein the exogenous HLA class I gene is a HLA-A, HLA-B, and/or HLA-C gene.

48. The method of claim 46, wherein the exogenous HLA class I gene is a gene from a human cancer patient.

49. The method of claim 48, wherein the exogenous HLA class I gene was cloned from the subject using locus-specific primer pairs.

50. The method of claim 48, wherein the subject has not been HLA typed.

51. The method of claim 48, comprising at least a first expression construct for an exogenous HLA class I gene is constructed by Gibson assembly.

52. The method of claim 48, wherein the exogenous HLA class I gene expresses an HLA polypeptide that binds to cancer antigen.

53. The method of claim 46, wherein introducing the exogenous HLA class I gene comprises transfecting the cells with a plasmid vector or an episomal vector or transducing the cells with a viral vector.

54. The method of claim 53, wherein introducing the exogenous HLA class I gene comprises transducing the cells with a lentiviral vector.

55. The method of claim 46, wherein the exogenous HLA class I gene expresses an HLA polypeptide that binds to cancer antigen, the method further comprising loading the cells with the cancer cell antigen that binds to the HLA polypeptide.

56. The method of claim 55, wherein the cells are loaded with the cancer cell antigen polypeptide.

57. The method of claim 55, wherein the cells are loaded with a vector encoding the cancer cell antigen.

58. The method of claim 46, wherein the cells are cultured in the presence of polybrene.

59. The method of claim 58, wherein the cells are cultured in the presence of about 1 to 20 ug/mL, 1 to 10 ug/mL, 5 to 10 ug/mL, or about 8 ug/mL of polybrene.

60. The method of claim 55, further comprising a co-culture the cells with immune effector cells.

61. The method of claim 60, wherein the immune effector cells are primary cells.

62. The method of claim 61, wherein the immune effector cells comprise T-cell, NK-cells or NK/T-cells.

63. The method of claim 62, wherein the T-cell comprise CD8⁺ T-cells or CD4⁺ T-cells.

64. The method of claim 55, wherein the cells are in co-culture in a medium that comprises cytokines that stimulate activation and/or growth of immune effector cells.

65. The method of claim 64, wherein medium comprises IL-2 or IL-15.

66. The method of any of claims 35-65, wherein the cells are an adherent cell culture.

67. The method of any of claims 35-66, wherein the cells are immobilized.

68. The method of any of claims 35-67, wherein the cells have been inactivated.

69. The method of claim 55, further comprising a co-culture of the cells with cells that express a candidate TCR or candidate CAR molecule.

70. The method of claim 69, further comprising selecting a candidate TCR or CAR based on binding to the cancer cell antigen.

71. A composition comprising a CRISPR guide RNA targeting a consensus sequence shared between HLA-A, HLA-B, and HLA-C for use in the preparation of an HLA class-I_{neg} cell population in accordance with any of claims 1-31.

72. The composition of claim 71, wherein the consensus sequence shared between HLA-A, HLA-B, and HLA-C is GGCTACTACAACCAGAGCG (SEQ ID NO:1).


SEQUENCE LISTING (10 sequences)

SEQ ID NO: 1 - DNA, 19 bp, Artificial sequence
  Synthetic oligonucleotide
ggctactacaaccagagcg

SEQ ID NO: 2 - DNA, 24 bp, Artificial sequence
  Synthetic oligonucleotide
ggctactacaaccagagcgaggcc

SEQ ID NO: 3 - DNA, 46 bp, Artificial sequence
  Synthetic primer
cgcagtcagtgctctagagctagcggattctccccagacsccgagg

SEQ ID NO: 4 - DNA, 45 bp, Artificial sequence
  Synthetic primer
gtaatccagaggttgattgtcgacgcacaaggcagctgtctcaca

SEQ ID NO: 5 - DNA, 46 bp, Artificial sequence
  Synthetic primer
cgcagtcagtgctctagagctagcgcacccggactcarartctcct

SEQ ID NO: 6 - DNA, 45 bp, Artificial sequence
  Synthetic primer
gtaatccagaggttgattgtcgacgcccttttcaagctgtgagag

SEQ ID NO: 7 - DNA, 46 bp, Artificial sequence
  Synthetic primer
cgcagtcagtgctctagagctagcgttctccccagasgccgagatg

SEQ ID NO: 8 - DNA, 46 bp, Artificial sequence
  Synthetic primer
gtaatccagaggttgattgtcgacgcgtctcaggctttacaagyga

SEQ ID NO: 9 - DNA, 31 bp, Artificial sequence
  Synthetic oligonucleotide
cctgcgcggctactacaaccagagcgaggcc

SEQ ID NO: 10 - DNA, 31 bp, Artificial sequence
  Synthetic oligonucleotide
cctgcgcggctactacaaccagagcgaggac